(19) United States
(12) Patent Application Publication
(10) Pub. No.: US 2025/0044265 A1
(43) Pub. Date: Feb. 6, 2025

(54) DATA ANALYSIS SYSTEM AND COMPUTER PROGRAM

(71) Applicant: SHIMADZU CORPORATION, Kyoto (JP)

(72) Inventors: Tomohiro KAWASE, Kyoto (JP); Harunori KAWAI, Kyoto (JP)

(73) Assignee: SHIMADZU CORPORATION, Kyoto (JP)

(21) Appl. No.: 18/767,253

(22) Filed: Jul. 9, 2024

(30) Foreign Application Priority Data
Aug. 1, 2023 (JP) 2023-125407

(51) Int. Cl. G01N 30/86

(52) U.S. Cl. CPC G01N 30/8693; G01N 30/8637; G01N 30/8658

(57) ABSTRACT

An analysis data storage part that stores analysis data including actual measurement values of a plurality of analysis results obtained by executing analysis using a plurality of analysis conditions, wherein each of the actual measurement values are in association with corresponding analysis condition of the plurality of analysis conditions in the analysis data storage part;
a model formula storage part that stores a model formula that includes an analysis condition as a variable and serves as a basis for a prediction formula for calculating a predicted value of an analysis result from an analysis condition; and
a data processor that performs arithmetic processing by using the analysis data stored in the analysis data storage part and the model formula stored in the model formula storage part, wherein
the data processor includes:
   a prediction formula generator configured to generate the prediction formula for the sample by applying the actual measurement values, together with respective corresponding analysis condition, of the plurality of analysis results stored in the analysis data storage part to the model formula stored in the model formula storage part; and
   a correlation data generator configured to generate correlation data indicating a correlation between predicted values, calculated by using the prediction formula generated by the prediction formula generator, of analysis results obtained by using two or more analysis conditions and actual measurement values of the analysis results obtained by using the two or more analysis conditions.

### BACKGROUND OF THE INVENTION

### 1. Field of the Invention

The present invention relates to a data analysis system and a computer program used for search of optimum gradient analysis for a sample, and the like.

### 2. Description of the Related Art

In a case where an analysis method (set of analysis conditions such as a mobile phase flow rate, column temperature, and a gradient condition) of liquid chromatography analysis is developed for a certain sample, the user himself or herself generally performs work of creating a plurality of analysis methods having different analysis conditions such as a gradient condition, and searching for an analysis method in which each peak is most separated and each peak component is eluted early in a chromatogram obtained by analyzing a sample by using each analysis method.

On the other hand, there is also software for assisting search for an optimum analysis condition for analysis of a target sample. Such software has a function of creating a prediction formula indicating a correlation between an analysis condition and an analysis result (retention time and peak width) for each component in a target sample from a chromatogram acquired by analyzing the target sample under a plurality of analysis conditions, predicting an optimum analysis condition for analysis of the target sample based on the created prediction formula for each component, and presenting the optimum analysis condition to the user.

### PRIOR ART DOCUMENTS

### Non Patent Documents

Non Patent Document 1: Weiqiang Hao, et. al., Computer aided optimization of multilinear gradient elution in liquid chromatography, Journal of Chromatography A, Volume 1635, 2021, 461754, ISSN 0021-9673,
Non Patent Document 2: P. J. Schoenmakers, et. al., Gradient selection in reversed-phase liquid chromatography J. Chromatogr., 149 (C) (1978), pp. 519-537
Non Patent Document 3: M. A. Quarry, et. al., Prediction of precise isocratic retention data from two or more gradient elution runs. Analysis of some associated errors Anal. Chem., 58 (4) (1986), pp. 907-917
Non Patent Document 4: S. R. Gallant, et. al., Modeling gradient elution of proteins in ion-exchange chromatography AIChE J, 42 (9) (1996), pp. 2511-2520
Non Patent Document 5: J. J. Baeza-Baeza, et. al., Approaches to model the retention and peak profile in linear gradient reversed-phase liquid chromatography J. Chromatogr. A, 1284 (2013), pp. 28-35
Non Patent Document 6: U. D. Neue, et. al., Peak compression in reversed-phase gradient elution J. Chromatogr. A, 1111 (2006), pp. 32-39
Non Patent Document 7: Weiqiang Hao, et. al., Influence of the pre-elution of solute in initial mobile phase on retention time and peak compression under linear gradient elution, Journal of Chromatography A, Volume 1618, 2020, 460858, ISSN 0021-9673,
Non Patent Document 8: H. Poppe, et. al., Peak width in solvent-programmed chromatography I. General description of peak broadening in solvent-programmed elution J. Chromatogr., 204 (1981), pp. 77-84

### SUMMARY OF THE INVENTION

In the prediction of an optimum analysis condition by the software, depending on an analysis condition and an analysis result used to create a prediction formula, there are a case where a highly reliable prediction formula is created and a case where a less reliable prediction formula is created. When an optimum analysis condition for analysis of a target sample is predicted based on an inaccurate prediction formula with low reliability, there is a possibility that an analysis condition that is not actually appropriate is predicted as an optimum analysis condition, and there is also a low possibility that a chromatogram expected by the user can be obtained if analysis is performed under a predicted analysis condition. Conversely, when an optimum analysis condition for analysis of a target sample is predicted based on an accurate prediction formula with high reliability, there is a high possibility that a predicted analysis condition is actually optimum, and there is also a high possibility that a chromatogram expected by the user can be obtained if analysis is performed under a predicted analysis condition.

However, there is no material for determining whether a created prediction formula has high reliability or low reliability, and it has been difficult for the user to determine whether or not an analysis condition predicted to be optimum for analysis of a target sample is actually optimum.

The present invention has been made in view of the above problem, and an object of the present invention is to provide the user with a material for determining reliability of a prediction formula.

A data analysis system according to the present invention includes:
an analysis data storage part that stores analysis data including actual measurement values of a plurality of analysis results obtained by executing analysis using a plurality of analysis conditions, wherein each of the actual measurement values are in association with corresponding analysis condition of the plurality of analysis conditions in the analysis data storage part;
a model formula storage part that stores a model formula that includes an analysis condition as a variable and serves as a basis for a prediction formula for calculating a predicted value of an analysis result from an analysis condition; and a data processor that performs arithmetic processing by using the analysis data stored in the analysis data storage part and the model formula stored in the model formula storage part, wherein
the data processor includes:
   a prediction formula generator configured to generate the prediction formula for the sample by applying the actual measurement values, together with respective corresponding analysis condition, of the plurality of analysis results stored in the analysis data storage part to the model formula stored in the model formula storage part; and
   a correlation data generator configured to generate correlation data indicating a correlation between predicted values, calculated by using the prediction formula generated by the prediction formula generator, of analysis results obtained by using two or more analysis conditions and actual measurement values of the analysis results obtained by using the two or more analysis conditions.

According to the data analysis system of the present invention, correlation data indicating a correlation between a predicted value of an analysis result and an actual measurement value of an analysis result for each analysis condition calculated by using a prediction formula is generated. Accordingly, the generated correlation data can be used as a material for determining reliability of the prediction formula. By the above, it is possible to provide the user with a material for determining reliability of a prediction formula.

### BRIEF DESCRIPTION OF THE DRAWINGS

FIG. 1 is a block diagram illustrating an embodiment of a configuration of a data analysis system;

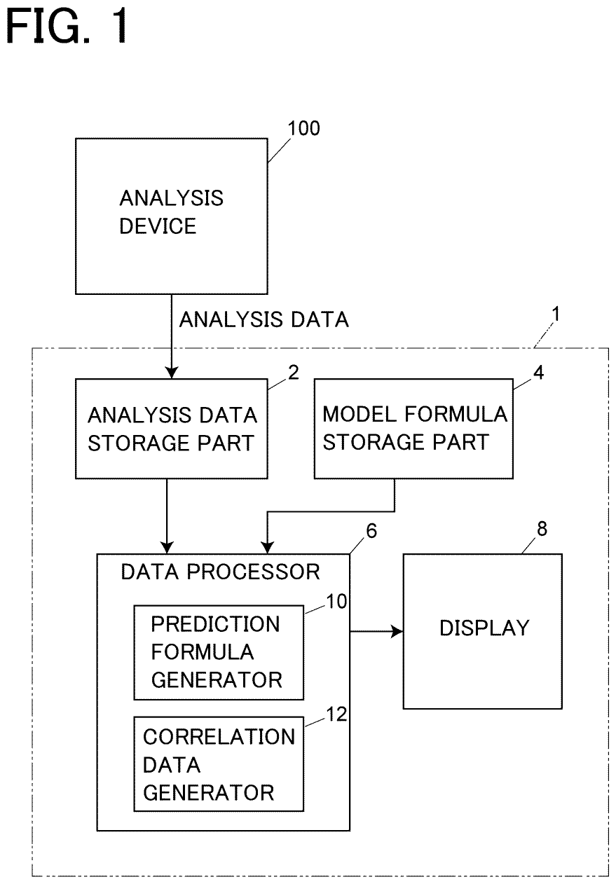

FIG. 2 is a diagram illustrating an example of a screen for displaying reliability information; and

FIG. 3 is a flowchart illustrating an example of data analysis processing performed in the embodiment.

### DETAILED DESCRIPTION OF THE INVENTION

Hereinafter, an embodiment of a data analysis system will be described with reference to the drawings.

A data analysis system 1 is constructed by introducing a computer program into a computer device, and includes an analysis data storage part 2, a model formula storage part 4, a data processor 6, and a display 8.

The analysis data storage part 2 is a storage area for storing, as analysis data, a plurality of analysis results and gradient conditions obtained by analyzing a sample by using a plurality of gradient conditions different from each other in an analysis device 100 in association with each other. The analysis result includes chromatogram information on retention time of each component in a sample and peak width of a peak of each component. The gradient condition is a condition of how a ratio of solvent constituting a mobile phase is changed over time. Note that, in this embodiment, the gradient condition will be described as an example of an analysis condition, but the present invention is not limited to the gradient condition.

The model formula storage part 4 stores a model formula based on a retention time prediction formula indicating a relationship between a gradient condition and retention time for each component in a sample, and a model formula that serves as a basis for a peak width prediction formula indicating a relationship between a gradient condition and peak width.

The analysis data storage part 2 and the model formula storage part 4 are realized by a partial area of an information storage device such as a hard disk drive. The analysis device 100 is, for example, a liquid chromatograph.

The data processor 6 includes a prediction formula generator 10 and a correlation data generator 12. The data processor 6 is realized by a computer circuit including a central processor (CPU), and the prediction formula generator 10 and the correlation data generator 12 are functions obtained by execution of a computer program in the data processor 6.

The prediction formula generator 10 is configured to generate a retention time prediction formula and a peak width prediction formula for each component in a sample by executing statistical analysis (fitting) by applying chromatogram information of the sample read from the analysis data storage part 2 to a model formula read from the model formula storage part 4. As an algorithm of statistical analysis, Bayesian estimation or the like can be used.

Here, a model formula will be described.

First, an example of a model formula that serves as a basis for retention coefficient prediction formula will be described.

A moving speed of a compound can be expressed by the reciprocal of a retention coefficient, and a relationship between retention time (t_{R}) and a retention coefficient k(t) at each time can be expressed by [Formula 1] below (see Non Patent Document 1).

$${t}_{0} = \frac{{t}_{D}}{k ( 0 )} + {\int }_{0}^{{t}_{R} - {t}_{0} - {t}_{D}} \frac{1}{k ( t )} dt \qquad [ Formula 1 ]$$

Here, tₚ is Dwell Time (data import time), and to is holdup time (time from the time of sample introduction until a vertex of a peak of a component not held in a column appears). A coefficient k(0) represents a retention coefficient in mobile phase composition before time 0. Here, it is assumed that, until analysis is started, mobile phase composition is fixed, and a retention coefficient does not change.

Further, it is known that a relationship between organic solvent concentration and a retention coefficient at the time of reverse phase analysis in a liquid chromatograph can be expressed by [Formula 2] below (see Non Patent Documents 1-5).

$$ln k ( t ) = ln {k}_{0} - {S}_{1} ϕ ( t ) + {S}_{2} {\{ ϕ ( t ) \}}^{2} \qquad [ Formula 2 ]$$

Here, k(t) is a retention coefficient at each time, φ(t) is an organic solvent ratio of a mobile phase at each time, and k₀ is a retention coefficient in a case where an organic solvent ratio is zero. S₁ and S₂ are coefficients, and are values that change depending on a solute, a stationary phase, and a mobile phase.

The prediction formula generator 10 can generate a retention time prediction formula of each component in a sample by obtaining k₀, S₁, and S₂ for each component in the sample by performing fitting by applying chromatogram information and gradient information of the sample to the above model formulas [Formula 1] and [Formula 2].

Next, an example of a model formula that serves as a basis for a peak width prediction formula will be described.

In reverse phase gradient analysis, peak width tends to decrease with respect to magnitude of retention time, and this phenomenon is called peak compression. The peak compression can be expressed by [Formula 3] below by using a compression coefficient G (see Non Patent Document 6).

$$W = 4 {Gt}_{0} ( 1 + k ( {t}_{R} ) ) / \sqrt{N} \qquad [ Formula 3 ]$$

Here, W is peak width, to is holdup time, k(t_{R}) is a retention coefficient at the time of elution, and N is a theoretical plate number of a column.

The retention coefficient k(t) can be obtained by the same algorithm as generation of a retention coefficient prediction formula. However, peak width varies greatly as compared with retention time, and may be excessively adapted to noise. For this reason, the prediction formula generator 10 can obtain k₀ and S₁ by performing fitting by applying chromatogram information and gradient information to [Formula 4] below ignoring a quadratic term of [Formula 2] and obtain a peak width prediction formula of each component in a sample.

$$ln k ( t ) = ln {k}_{0} - {S}_{1} ϕ ( t ) \qquad [ Formula 4 ]$$

Note that the compression coefficient G can be calculated by Formula [5] below using a retention coefficient (see Non Patent Documents 7-8).

$${G}^{2} = \frac{{k ( {t}_{R} )}^{2}}{{t}_{0} ( 1 + {k ( {t}_{R} )}^{2} )} \{ \frac{{t}_{D} ( 1 + {k ( 0 )}^{3} )}{{k ( 0 )}^{3}} + {\int }_{0}^{{t}_{R} - {t}_{0} - {t}_{D}} \frac{{( 1 + k ( t ) )}^{2}}{{k ( t )}^{3}} dt \} \qquad [ Formula 5 ]$$

The correlation data generator 12 is configured to calculate a predicted value of retention time and a predicted value of peak width for each gradient condition of each component in a sample by using a retention time prediction formula and a peak width prediction formula generated based on the above-described model formula, and generate correlation data indicating a correlation between each of the calculated predicted values and an actual measurement value (a value of retention time and a value of peak width in analysis data) under a corresponding gradient condition.

The display 8 is connected to the data processor 6, and for example, when desired by the user, correlation data generated by the correlation data generator 12 is displayed on the display 8.

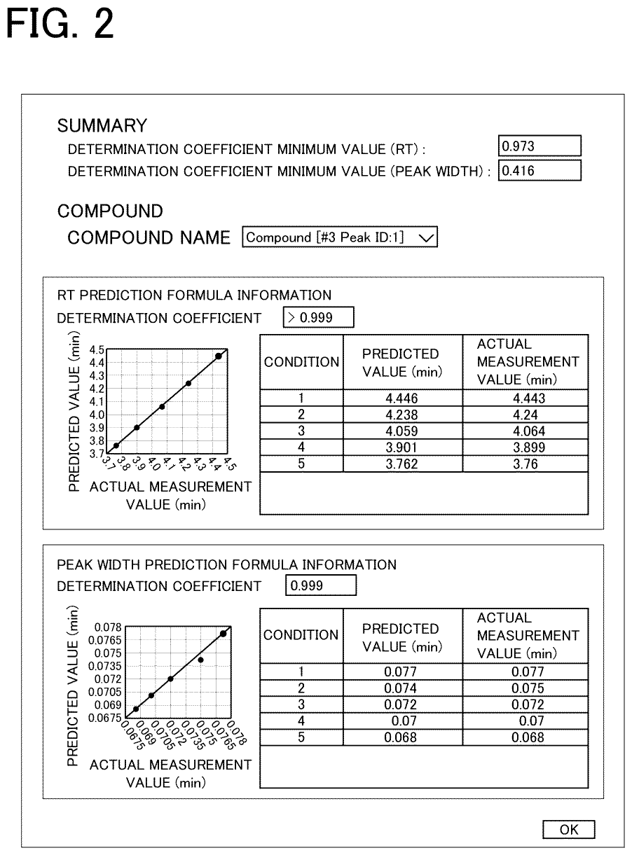

FIG. 2 illustrates an example of correlation data displayed on the display 8.

The correlation data illustrated in FIG. 2 is for one component (compound) designated by the user. The correlation data includes retention time (RT) prediction formula information and peak width prediction formula information for each component in a sample.

Each of retention time prediction formula information and peak width prediction formula information includes a two-dimensional graph in which one of two axes orthogonal to each other is an axis of an actual measurement value for each gradient condition and the other axis is an axis of a predicted value for each gradient condition, a table showing a predicted value and an actual measurement value for each gradient condition, and a determination coefficient for each prediction formula.

Each plot on the two-dimensional graph indicates an intersection of a predicted value and an actual measurement value under each gradient condition. In a case where a prediction formula is matches under all gradient conditions (a predicted value takes a value close to an actual measurement value=appropriate), a plot of an actual measurement value is located on a straight line of x=y with a slope of one in the two-dimensional graph. On the other hand, in a case where there exists a gradient condition to which a prediction formula does not apply (not appropriate), there exists a plot of an actual measurement value deviating from a straight line of x=y in the two-dimensional graph. As described above, when the two-dimensional graph is viewed, it clearly shows under which gradient condition a prediction formula has high reliability and under which gradient condition a prediction formula has low reliability, and the user can easily recognize reliability of a prediction formula. Furthermore, since tables of a predicted value and an actual measurement value for each gradient condition are shown at the same time, the user can easily check how much a predicted value deviates from an actual measurement value.

Further, a “determination coefficient” indicated in each of retention time prediction formula information and peak width prediction formula information is a statistic for evaluating whether or not a prediction formula is appropriate. As such a statistic, an Rhat statistic is known. An Rhat statistic is approximately 1 if estimation of a prediction formula is appropriate. If the Rhat statistic exceeds 1.1, it is generally evaluated that estimation is performed but the estimation is not appropriate. Rhat is a value obtained in a case where Bayesian estimation is used as a statistical analysis algorithm of fitting used for generating a prediction formula. This statistic is 1 when variation of an actual measurement value can be completely explained by a predicted value obtained by a prediction formula, and is a value smaller than 1 as the number of variations that cannot be explained is larger (as prediction performance of the prediction formula is worse). The user can also check reliability of each prediction formula by referring to such a numerical value.

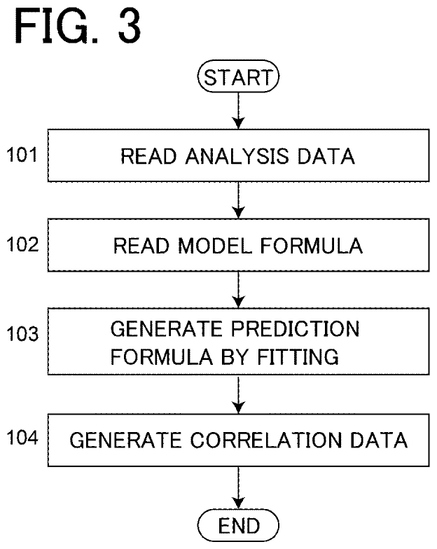

Next, data analysis processing executed by the data processor 6 will be described with reference to a flowchart of FIG. 3.

The prediction formula generator 10 of the data processor 6 reads analysis data of a sample to be analyzed from the analysis data storage part 2 (Step 101), and further reads a model formula used for generating a prediction formula from the model formula storage part 4 (Step 102). After the above, the data processor 6 determines each coefficient of a model formula by applying analysis data to the model formula and performing fitting using a predetermined statistical analysis algorithm, and generates a retention time prediction formula and a peak width prediction formula (Step 103).

When the retention time prediction formula and the peak width prediction formula are generated, predicted values of retention time and peak width for each gradient condition are calculated using the generated prediction formulas, and correlation data as shown in FIG. 2 is generated using the calculated predicted values and an actual measurement value (Step 104). The correlation data generated here is displayed on the display 8, for example, when desired by the user.

Note that the example described above is merely an example of an embodiment of the present invention. As for an analysis condition, correlation data for each gradient condition is described. However, the present invention is not limited to this, and other analysis conditions (column oven temperature, a flow rate, and the like) can be similarly applied. The embodiment of the data analysis system and the computer program according to the present invention is as described below.

The embodiment of the data analysis system according to the present invention includes:
an analysis data storage part that stores analysis data including actual measurement values of a plurality of analysis results obtained by executing analysis using a plurality of analysis conditions, wherein each of the actual measurement values are in association with corresponding analysis condition of the plurality of analysis conditions in the analysis data storage part;
a model formula storage part that stores a model formula that includes an analysis condition as a variable and serves as a basis for a prediction formula for calculating a predicted value of an analysis result from an analysis condition; and
a data processor that performs arithmetic processing by using the analysis data stored in the analysis data storage part and the model formula stored in the model formula storage part, wherein
the data processor includes:
   a prediction formula generator configured to generate the prediction formula for the sample by applying the actual measurement values, together with respective corresponding analysis condition, of the plurality of analysis results stored in the analysis data storage part to the model formula stored in the model formula storage part; and
   a correlation data generator configured to generate correlation data indicating a correlation between predicted values, calculated by using the prediction formula generated by the prediction formula generator, of analysis results obtained by using two or more analysis conditions and actual measurement values of the analysis results obtained by using the two or more analysis conditions.

In an aspect [1] of the embodiment, the correlation data includes a two-dimensional graph in which one of two axes intersecting each other is an axis of an actual measurement value of the analysis result for each of the analysis conditions and another axis is an axis of a predicted value of the analysis result for each of the analysis conditions.

In an aspect [2] of the embodiment, the correlation data includes reliability information of the prediction formula in consideration of a deviation degree between an actual measurement value of the analysis result and a predicted value of the analysis result for a plurality of the analysis conditions. This aspect [2] can be combined with the above aspect [1].

In an aspect [3] of the embodiment, the analysis result includes retention time of each component contained in the sample. This aspect [3] can be combined with the above aspects [1] and/or [2].

In an aspect [4] of the embodiment, the analysis result includes a peak width of each component included in the sample. This aspect [4] can be combined with the above aspects [1], [2], and/or [3].

An aspect [5] of the embodiment further includes a display (8), and the data processor (6) is configured to display the correlation data generated by the correlation data generator (12) on the display (8). This aspect [5] can be combined with the above aspects [1], [2], [3], and/or [4].

In an aspect [6] of the embodiment, the two or more analysis conditions can include an analysis condition other than a plurality of the analysis conditions. According to such an aspect, it is possible to check whether or not prediction of an analysis result under an analysis condition other than an analysis condition used for generating a prediction formula is appropriate. This aspect [6] can be combined with the above aspects [1], [2], [3], [4], and/or [5].

An embodiment of a computer program according to the present invention is configured to construct the above-described data analysis system by being introduced into a computer.

### DESCRIPTION OF REFERENCE SIGNS

1 data analysis system
2 data storage part
4 data processor
6 information input device
8 display

What is claimed is:

1. A data analysis system comprising:
an analysis data storage part that stores analysis data including actual measurement values of a plurality of analysis results obtained by executing analysis using a plurality of analysis conditions, wherein each of the actual measurement values are in association with corresponding analysis condition of the plurality of analysis conditions in the analysis data storage part;
a model formula storage part that stores a model formula that includes an analysis condition as a variable and serves as a basis for a prediction formula for calculating a predicted value of an analysis result from an analysis condition; and
a data processor that performs arithmetic processing by using the analysis data stored in the analysis data storage part and the model formula stored in the model formula storage part, wherein
the data processor includes:
a prediction formula generator configured to generate the prediction formula for the sample by applying the actual measurement values, together with respective corresponding analysis condition, of the plurality of analysis results stored in the analysis data storage part to the model formula stored in the model formula storage part; and
a correlation data generator configured to generate correlation data indicating a correlation between predicted values, calculated by using the prediction formula generated by the prediction formula generator, of analysis results obtained by using two or more analysis conditions and actual measurement values of the analysis results obtained by using the two or more analysis conditions.

2. The data analysis system according to claim 1, wherein the correlation data includes a two-dimensional graph in which one of two axes intersecting each other is an axis of an actual measurement value of the analysis result for each of the analysis conditions and another axis is an axis of a predicted value of the analysis result for each of the analysis conditions.

3. The data analysis system according to claim 1, wherein the correlation data includes reliability information of the prediction formula in consideration of a deviation degree between an actual measurement value of the analysis result and a predicted value of the analysis result for the plurality of analysis conditions.

4. The data analysis system according to claim 1, wherein the analysis result includes retention time of each component contained in the sample.

5. The data analysis system according to claim 1, wherein the analysis result includes a peak width of each component included in the sample.

6. The data analysis system according to claim 1, further comprising a display, wherein
the data processor is configured to display the correlation data generated by the correlation data generator on the display.

7. The data analysis system according to claim 1, wherein the two or more analysis conditions include an analysis condition other than the plurality of analysis conditions.

8. A computer program configured to construct the data analysis system according to claim 1 by being introduced into a computer.
